(19) United States
(12) Patent Application Publication
(10) Pub. No.: US 2023/0125439 A1
(43) Pub. Date: Apr. 27, 2023

(54) PERCUTANEOUS CIRCULATORY SUPPORT DEVICE FACILITATING REDUCED HEMOLYSIS

(71) Applicant: Boston Scientific Scimed, Inc., Maple Grove, MN (US)

(72) Inventors: Anthony Malone, Galway (IE); Olena Pernatiy, Galway (IE); Javier Palomar-Moreno, Galway (IE)

(21) Appl. No.: 17/974,844

(22) Filed: Oct. 27, 2022

Related U.S. Application Data
(60) Provisional application No. 63/272,488, filed on Oct. 27, 2021.

(51) Int. Cl. A61M 60/226; A61M 60/165; A61M 60/419; A61M 60/806

(52) U.S. Cl. CPC A61M 60/226; A61M 60/165; A61M 60/419; A61M 60/806

(57) ABSTRACT

A percutaneous circulatory support device includes an impeller housing and a primary impeller disposed within the impeller housing. The primary impeller is rotatable relative to the impeller housing to cause blood to flow through the percutaneous circulatory support device. The device further includes a counter impeller disposed within the impeller housing.

### CROSS REFERENCE TO RELATED APPLICATION

This application claims priority to Provisional Application No. 63/272,488, filed Oct. 27, 2021, which is herein incorporated by reference in its entirety.

### TECHNICAL FIELD

The present disclosure relates to percutaneous circulatory support systems. More specifically, the disclosure relates to percutaneous circulatory support devices that facilitate reduced hemolysis and/or related complications.

### BACKGROUND

Percutaneous circulatory support devices can provide transient support for up to approximately several weeks in patients with compromised heart function or cardiac output. Operation of such devices, however, may cause some amount of hemolysis (that is, the rupture or destroying of blood cells) or other complications. Hemolysis can in turn cause thrombi formation or other complications. Accordingly, there is a need for improved devices that facilitate reduced hemolysis and/or related complications.

### SUMMARY

In an Example 1, a percutaneous circulatory support device comprises an impeller housing; a primary impeller disposed within the impeller housing, the primary impeller being rotatable relative to the impeller housing to cause blood to flow through the percutaneous circulatory support device; and a counter impeller disposed within the impeller housing.

In an Example 2, the percutaneous circulatory support device of Example 1, further comprising a motor operatively coupled to the primary impeller and rotating the primary impeller relative to the impeller housing to cause blood to flow through the percutaneous circulatory support device.

In an Example 3, the percutaneous circulatory support device of Example 2, wherein the impeller housing comprises a proximal end portion, and the motor rotates the counter impeller relative to the impeller housing to create a turbulent barrier and thereby cause blood to flow away from the proximal end portion of the housing.

In an Example 4, the percutaneous circulatory support device of Example 3, wherein the motor rotates the primary impeller and the counter impeller together relative to the impeller housing.

In an Example 5, the percutaneous circulatory support device of any of Examples 1 to 4, further comprising an impeller shaft being fixed relative to the primary impeller and the counter impeller.

In an Example 6, the percutaneous circulatory support device of Example 4, further comprising a driving magnet operatively coupled to the motor and a driven magnet operatively coupled to the driving magnet, and the motor rotates the primary impeller and the counter impeller together, via the driving magnet and the driven magnet, relative to the impeller housing.

In an Example 7, the percutaneous circulatory support device of Example 6, wherein the counter impeller is disposed between the primary impeller and the driven magnet.

In an Example 8, the percutaneous circulatory support device of either of Examples 6 or 7, wherein the driven magnet is disposed in the proximal end portion of the impeller housing.

In an Example 9, the percutaneous circulatory support device of any of Examples 1 to 8, wherein the primary impeller comprises a first number of impeller blades and the counter impeller comprises a second number of impeller blades, the second number of impeller blades being greater than the first number of impeller blades.

In an Example 10, the percutaneous circulatory support device of any of Examples 1 to 8, wherein the primary impeller comprises a plurality of first impeller blades and the counter impeller comprises a plurality of second impeller blades, the second impeller blades having the opposite pitch than the first impeller blades.

In an Example 11, the percutaneous circulatory support device of any of Examples 1 to 10, wherein the impeller housing further comprises an outlet, and the outlet extends both proximally and distally beyond the counter impeller.

In an Example 12, the percutaneous circulatory support device of Examples 1 to 11, wherein the primary impeller has a first axial length, the counter impeller has a second axial length, and the second axial length is less than the first axial length.

In an Example 13, a percutaneous circulatory support device comprises an impeller housing; a first impeller disposed within the impeller housing, the first impeller being rotatable relative to the impeller housing to cause blood to flow through the percutaneous circulatory support device; and a second impeller disposed within the impeller housing.

In an Example 14, the percutaneous circulatory support device of Example 13, further comprising a motor operatively coupled to the first impeller and rotating the first impeller relative to the impeller housing to cause blood to flow through the percutaneous circulatory support device.

In an Example 15, the percutaneous circulatory support device of either of Examples 13 or 14, wherein the impeller housing comprises a proximal end portion, and the second impeller is rotatable relative to the impeller housing to create a turbulent barrier and thereby cause blood to flow away from the proximal end portion of the housing.

In an Example 16, a percutaneous circulatory support device comprises an impeller housing comprising a proximal end portion; a primary impeller disposed within the impeller housing, the primary impeller being rotatable relative to the impeller housing to cause blood to flow through the percutaneous circulatory support device; and a counter impeller disposed within the impeller housing, the counter impeller being rotatable relative to the impeller housing to create a turbulent barrier and thereby cause blood to flow away from the proximal end portion of the housing.

In an Example 17, the percutaneous circulatory support device of Example 16, further comprising a motor operatively coupled to the primary impeller and rotating the primary impeller relative to the impeller housing to cause blood to flow through the percutaneous circulatory support device.

In an Example 18, the percutaneous circulatory support device of Example 17, wherein the motor is further operatively coupled to the counter impeller, the motor rotating the primary impeller and the counter impeller together relative to the impeller housing.

In an Example 19, the percutaneous circulatory support device of Example 18, further comprising an impeller shaft being fixed relative to the primary impeller and the counter impeller.

In an Example 20, the percutaneous circulatory support device of Example 18, further comprising a driving magnet operatively coupled to the motor and a driven magnet operatively coupled to the driving magnet, and the motor rotates the primary impeller and the counter impeller together, via the driving magnet and the driven magnet, relative to the impeller housing.

In an Example 21, the percutaneous circulatory support device of Example 20, wherein the counter impeller is disposed between the primary impeller and the driven magnet.

In an Example 22, the percutaneous circulatory support device of Example 20, wherein the driven magnet is disposed in the proximal end portion of the impeller housing.

In an Example 23, the percutaneous circulatory support device of Example 16, wherein the primary impeller comprises a first number of impeller blades and the counter impeller comprises a second number of impeller blades, the second number of impeller blades being greater than the first number of impeller blades.

In an Example 24, the percutaneous circulatory support device of Example 16, wherein the primary impeller comprises a plurality of first impeller blades and the counter impeller comprises a plurality of second impeller blades, the second impeller blades having the opposite pitch than the first impeller blades.

In an Example 25, the percutaneous circulatory support device of Example 16, wherein the impeller housing further comprises an outlet, and the outlet extends both proximally and distally beyond the counter impeller.

In an Example 26, the percutaneous circulatory support device of Example 16, wherein the primary impeller has a first axial length, the counter impeller has a second axial length, and the second axial length is less than the first axial length.

In an Example 27, a percutaneous circulatory support device comprises an impeller housing comprising a proximal end portion; a first impeller disposed within the impeller housing, the first impeller being rotatable relative to the impeller housing to cause blood to flow through the percutaneous circulatory support device; and a second impeller disposed within the impeller housing, the second impeller being rotatable relative to the impeller housing to create a turbulent barrier and thereby cause blood to flow away from the proximal end portion of the housing.

In an Example 28, the percutaneous circulatory support device of Example 27, further comprising a motor operatively coupled to the first impeller and rotating the first impeller relative to the impeller housing to cause blood to flow through the percutaneous circulatory support device.

In an Example 29, the percutaneous circulatory support device of Example 28, wherein the motor is further operatively coupled to the second impeller, the motor rotating the first impeller and the second impeller together relative to the impeller housing.

In an Example 30, the percutaneous circulatory support device of Example 29, further comprising a driving magnet operatively coupled to the motor and a driven magnet operatively coupled to the driving magnet, and the motor rotates the first impeller and the second impeller together, via the driving magnet and the driven magnet, relative to the impeller housing.

In an Example 31, the percutaneous circulatory support device of Example 29, further comprising an impeller shaft being fixed relative to the first impeller and the second impeller.

In an Example 32, a method for using a percutaneous circulatory support device comprises: positioning the percutaneous circulatory support device at a target location within a patient; rotating a primary impeller of the percutaneous circulatory support device to cause blood to flow through the percutaneous circulatory support device; and rotating a counter impeller of the percutaneous circulatory support device to create a turbulent barrier and thereby cause blood to flow away from a proximal end portion of a housing of the percutaneous circulatory support device.

In an Example 33, the method of Example 32, wherein rotating the primary impeller and rotating the counter impeller comprise rotating the primary impeller and the counter impeller together relative to the housing.

In an Example 34, the method of Example 33, wherein the percutaneous circulatory support device further comprises a motor operatively coupled to the primary impeller and the counter impeller, and rotating the primary impeller and the counter impeller together relative to the housing comprises driving the primary impeller and the counter impeller via the motor.

In an Example 35, the method of Example 34, wherein the percutaneous circulatory support device further comprises a driving magnet operatively coupled to the motor and a driven magnet operatively coupled to the driving magnet, and driving the primary impeller and the counter impeller via the motor comprises driving the first impeller and the second impeller together, via the motor, the driving magnet, and the driven magnet, relative to the housing.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a side sectional view of an illustrative mechanical circulatory support device (also referred to herein, interchangeably, as a “blood pump”), in accordance with embodiments of the subject matter disclosed herein.

FIG. 2 is a perspective view of the mechanical circulatory support device of FIG. 1, with housing components of the device shown in phantom lines, in accordance with embodiments of the subject matter disclosed herein.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

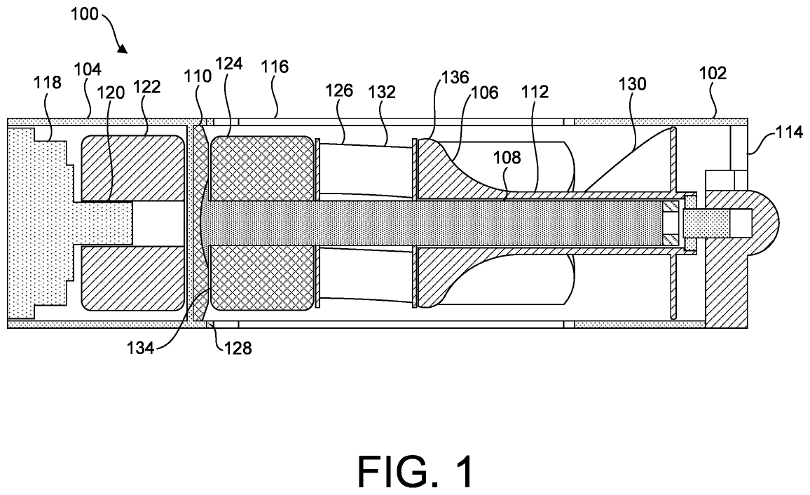

FIG. 1 depicts a partial side sectional view of an illustrative mechanical circulatory support device 100 (also referred to herein, interchangeably, as a “blood pump”) in accordance with embodiments of the subject matter disclosed herein. The device 100 may form part of a percutaneous circulatory support system, together with a guidewire and an introducer sheath (not shown). More specifically, the guidewire and the introducer sheath may facilitate percutaneously delivering the device 100 to a target location within a patient, such as within the patient's heart. Alternatively, the device 100 may be delivered to a different target location within a patient.

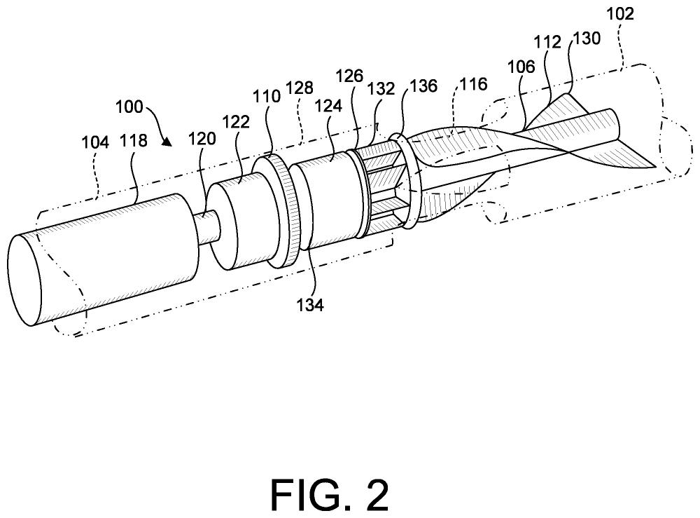

With continued reference to FIG. 1 and additional reference to FIG. 2, the device 100 generally includes an impeller housing 102 and a motor housing 104. In some embodiments, the impeller housing 102 and the motor housing 104 may be integrally or monolithically constructed. In other embodiments, the impeller housing 102 and the motor housing 104 may be separate components configured to be removably or permanently coupled.

The impeller housing 102 carries an impeller assembly 106 therein. The impeller assembly 106 includes an impeller shaft 108 (FIG. 1) that is rotatably supported by at least one bearing, such as a bearing 110. The impeller assembly 106 also includes a primary or first primary impeller 112 that rotates relative to the impeller housing 102 to drive blood through the device 100. More specifically, the primary impeller 112 causes blood to flow from a blood inlet 114 (FIG. 1) formed on the impeller housing 102, through the impeller housing 102, and out of a blood outlet 116 formed on the impeller housing 102. In some embodiments and as illustrated, the impeller shaft 108 and the primary impeller 112 may be separate components, and in other embodiments the impeller shaft 108 and the primary impeller 112 may be integrated. In some embodiment and as illustrated, the inlet 114 and/or the outlet 116 may each include multiple apertures. In other embodiments, the inlet 114 and/or the outlet 116 may each include a single aperture. In some embodiments and as illustrated, the inlet 114 may be formed on an end portion of the impeller housing 102 and the outlet 116 may be formed on a side portion of the impeller housing 102. In other embodiments, the inlet 114 and/or the outlet 116 may be formed on other portions of the impeller housing 102. In some embodiments, the impeller housing 102 may couple to a distally extending cannula (not shown), and the cannula may receive and deliver blood to the inlet 114.

With continued reference to FIGS. 1 and 2, the motor housing 104 carries a motor 118, and the motor 118 is configured to rotatably drive the primary impeller 112 relative to the impeller housing 102. In the illustrated embodiment, the motor 118 rotates a drive shaft 120, which is coupled to a driving magnet 122. Rotation of the driving magnet 122 causes rotation of a driven magnet 124, which is connected to and rotates together with the impeller assembly 106. More specifically, in embodiments incorporating the impeller shaft 108, the impeller shaft 108 and the primary impeller 112 are configured to rotate with the driven magnet 124. In other embodiments, the motor 118 may couple to the impeller assembly 106 via other components.

In some embodiments, a controller (not shown) may be operably coupled to the motor 118 and configured to control the motor 118. In some embodiments, the controller may be disposed within the motor housing 104. In other embodiments, the controller may be disposed outside of the motor housing 104 (for example, in a catheter handle, an independent housing, etc.). In some embodiments, the controller may include multiple components, one or more of which may be disposed within the motor housing 104. According to embodiments, the controller may be, may include, or may be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more Central Processing Units (CPUs), software, hardware, firmware, or any combination of these and/or other components. Although the controller is referred to herein in the singular, the controller may be implemented in multiple instances, distributed across multiple computing devices, instantiated within multiple virtual machines, and/or the like. In other embodiments, the motor 118 may be controlled in other manners.

With further reference to FIGS. 1 and 2, the device 100 facilitates reduced device-induced hemolysis compared to conventional devices. More specifically, the device 100 includes a counter or second impeller 126 that is fixed relative to and configured to rotate with the impeller shaft 108, the primary impeller 112, and the driven magnet 124. As the counter impeller 126 rotates, it creates small eddies that form a turbulent barrier. This barrier causes blood to flow away from a proximal end portion 128 of the impeller housing 102 (that is, a portion of the housing 102 adjacent to the driven magnet 124). As a result, the counter impeller 126 inhibits blood from pooling at the proximal end portion 128 of the impeller housing 102, which reduces hemolysis and/or thrombosis. In some embodiments and as illustrated, the counter impeller 126 is disposed between the primary impeller 112 and the driven magnet 124. There are multiple designs for the counter impeller 126, including straight and curved blades. In some embodiments and as illustrated, the apertures of the outlet 116 extend both proximally and distally beyond the counter impeller 126. In some embodiments and as illustrated, the counter impeller 126 includes more blades than the primary impeller 112. Stated another way, the primary impeller 112 includes a first number of impeller blades 130, the counter impeller 126 comprises a second number of impeller blades 132, and the second number of impeller blades 132 is greater than the first number of impeller blades 130. In other embodiments, the counter impeller 126 and the primary impeller 112 include the same number of blades. In some embodiments and as illustrated, the blades 130 of the primary impeller 112 and the blades 132 of the counter impeller 126 have the opposite pitch. In some embodiments and as illustrated, the counter impeller 126 is axially shorter than the primary impeller 112. Stated another way, the primary impeller 112 has a first axial length (that is, a dimension in the longitudinal direction of the device 100), the counter impeller 126 has a second axial length, and the second axial length is less than the first axial length. In some embodiments and as illustrated, the impeller shaft 108 and the counter impeller 126 may be separate components, and in other embodiments the impeller shaft 108 and the counter impeller 126 may be integrated.

The device 100 may also include one or more additional features that facilitate reduced device-induced hemolysis compared to conventional devices. For example, the apertures of the outlet 116 may be relatively long compared to those of conventional devices. More specifically, the apertures of the outlet 116 may each extend to a proximal end 134 of the driven magnet 124. Such apertures inhibit blood from pooling in the proximal end portion 128 of the impeller housing 102, which reduces hemolysis and/or thrombosis. As another example, a proximal end portion 136 of the primary impeller 112 may have a flattened shape, in contrast to lips or peaks of the impellers of conventional devices. Such a shape inhibits blood from forming and pooling in vortices adjacent to the primary impeller 112.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A percutaneous circulatory support device, comprising:
an impeller housing comprising a proximal end portion;
a primary impeller disposed within the impeller housing, the primary impeller being rotatable relative to the impeller housing to cause blood to flow through the percutaneous circulatory support device; and
a counter impeller disposed within the impeller housing, the counter impeller being rotatable relative to the impeller housing to create a turbulent barrier and thereby cause blood to flow away from the proximal end portion of the housing.

2. The percutaneous circulatory support device of claim 1, further comprising a motor operatively coupled to the primary impeller and rotating the primary impeller relative to the impeller housing to cause blood to flow through the percutaneous circulatory support device.

3. The percutaneous circulatory support device of claim 2, wherein the motor is further operatively coupled to the counter impeller, the motor rotating the primary impeller and the counter impeller together relative to the impeller housing.

4. The percutaneous circulatory support device of claim 3, further comprising an impeller shaft being fixed relative to the primary impeller and the counter impeller.

5. The percutaneous circulatory support device of claim 3, further comprising a driving magnet operatively coupled to the motor and a driven magnet operatively coupled to the driving magnet, and the motor rotates the primary impeller and the counter impeller together, via the driving magnet and the driven magnet, relative to the impeller housing.

6. The percutaneous circulatory support device of claim 5, wherein the counter impeller is disposed between the primary impeller and the driven magnet.

7. The percutaneous circulatory support device of claim 5, wherein the driven magnet is disposed in the proximal end portion of the impeller housing.

8. The percutaneous circulatory support device of claim 1, wherein the primary impeller comprises a first number of impeller blades and the counter impeller comprises a second number of impeller blades, the second number of impeller blades being greater than the first number of impeller blades.

9. The percutaneous circulatory support device of claim 1, wherein the primary impeller comprises a plurality of first impeller blades and the counter impeller comprises a plurality of second impeller blades, the second impeller blades having the opposite pitch than the first impeller blades.

10. The percutaneous circulatory support device of claim 1, wherein the impeller housing further comprises an outlet, and the outlet extends both proximally and distally beyond the counter impeller.

11. The percutaneous circulatory support device of claim 1, wherein the primary impeller has a first axial length, the counter impeller has a second axial length, and the second axial length is less than the first axial length.

12. A percutaneous circulatory support device, comprising:
an impeller housing comprising a proximal end portion;
a first impeller disposed within the impeller housing, the first impeller being rotatable relative to the impeller housing to cause blood to flow through the percutaneous circulatory support device; and
a second impeller disposed within the impeller housing, the second impeller being rotatable relative to the impeller housing to create a turbulent barrier and thereby cause blood to flow away from the proximal end portion of the housing.

13. The percutaneous circulatory support device of claim 12, further comprising a motor operatively coupled to the first impeller and rotating the first impeller relative to the impeller housing to cause blood to flow through the percutaneous circulatory support device.

14. The percutaneous circulatory support device of claim 13, wherein the motor is further operatively coupled to the second impeller, the motor rotating the first impeller and the second impeller together relative to the impeller housing.

15. The percutaneous circulatory support device of claim 14, further comprising a driving magnet operatively coupled to the motor and a driven magnet operatively coupled to the driving magnet, and the motor rotates the first impeller and the second impeller together, via the driving magnet and the driven magnet, relative to the impeller housing.

16. The percutaneous circulatory support device of claim 15, further comprising an impeller shaft being fixed relative to the first impeller and the second impeller.

17. A method for using a percutaneous circulatory support device, comprising:
positioning the percutaneous circulatory support device at a target location within a patient;
rotating a primary impeller of the percutaneous circulatory support device to cause blood to flow through the percutaneous circulatory support device; and
rotating a counter impeller of the percutaneous circulatory support device to create a turbulent barrier and thereby cause blood to flow away from a proximal end portion of a housing of the percutaneous circulatory support device.

18. The method of claim 17, wherein rotating the primary impeller and rotating the counter impeller comprise rotating the primary impeller and the counter impeller together relative to the housing.

19. The method of claim 18, wherein the percutaneous circulatory support device further comprises a motor operatively coupled to the primary impeller and the counter impeller, and rotating the primary impeller and the counter impeller together relative to the housing comprises driving the primary impeller and the counter impeller via the motor.

20. The method of claim 19, wherein the percutaneous circulatory support device further comprises a driving magnet operatively coupled to the motor and a driven magnet operatively coupled to the driving magnet, and driving the primary impeller and the counter impeller via the motor comprises driving the first impeller and the second impeller together, via the motor, the driving magnet, and the driven magnet, relative to the housing.
